(19) United States
(12) Patent Application Publication
(10) Pub. No.: US 2021/0154050 A1
(43) Pub. Date: May 27, 2021

(54) DRESSING FOR A NURSING MOTHER

(71) Applicants: Jennifer Gloeckner Powers, Iowa City, IA (US); Mary Gloeckner Bouljon, Moline, IL (US)

(72) Inventors: Jennifer Gloeckner Powers, Iowa City, IA (US); Mary Gloeckner Bouljon, Moline, IL (US)

(21) Appl. No.: 17/103,174

(22) Filed: Nov. 24, 2020

Related U.S. Application Data
(60) Provisional application No. 62/941,544, filed on Nov. 27, 2019.

(51) Int. Cl. A61F 13/14; A61F 13/00

(52) U.S. Cl. CPC A61F 13/14; A61F 2013/00217; A61F 2013/00748; A61F 13/00063

(57) ABSTRACT

A dressing for the irritated breast of a nursing mother is described. The dressing is composed of hydrocolloid and non-adherent protective layers for covering the injured nipple and areola.

### CROSS-REFERENCE TO RELATED APPLICATION

This patent application claims the benefit of U.S. Provisional Patent Application No, 62/941,544, filed Nov. 27, 2019, entitled “DRESSING FOR NURSING MOTHER”, which is incorporated by reference herein in its entirety.

### BACKGROUND

At any given time, millions of women worldwide are breastfeeding and many of them struggle, developing skin trauma to their nipples in the form of abrasions, fissures, and even ulcers that can lead to failure to nurse, failure to thrive for the infants, psychological trauma for the mothers in the form of pain, anguish, anxiety and trauma for the whole family. The skin trauma is primarily related to frequent and strong sucking applied to such tender tissues along with the enzymes as well as bacteria and viruses present in human saliva though sheer forces from friction applied from the infant's gums and/or by budding teeth that may also play a role. These repetitive strain injuries have little time to heal because of the need for repetitive, frequent nursing. Studies have shown that scabs/blisters affect at least 60% of nursing mothers while more than 80% will struggle with pain, more often than not involving both nipples. Moreover, the psychological trauma to the mother is difficult if not impossible to resolve because of the conflicts resulting from the desire to nurse and at the same time move away from pain. Mother are often suffering, at home, in silence, and without the help of medical professionals. These problems do not get enough attention in medicine as obstetric and gynecologic providers are most often confronting life-threatening health crises, lactation consultants are unable to prescribe medications and lack certain medical knowledge, and pediatricians often identify issues but are unable to provide guidance. Dermatologists and wound healing expertise have been absent from this space. However, there have been calls-to-action in recent years to bring more innovative technologies into this space: https://www.maketthebreastpumpnotsuck.com/why-breastfeeding-innovation.

Current solutions are inadequate. Medical and non-medical advice suggest coating the breast and nipples with lanolin, petroleum jelly, poly-antibiotic ointments, expressed breastmilk, and/or reverting to bottle feeding until the breast heals. Lanolin may cause allergic dermatitis; antibiotic ointments may be unnecessary for a problem that is more repetitive strain/friction/pressure associated than at its core an infectious problem. Refraining from breast-feeding defeats the objective of breast-feeding in several ways including but not limited to failure to deliver to the infant serum protections from the mother. Current dressings are soothing but are too adhesive, contain allergens and irritants and do not heal full-thickness skin loss. For example, over-the-counter Band-Aid® Hydro Seal® are affordable products but they create full surface area adherence, which is potentially damaging to fresh keratinocytes on a healing injured nipple (FIG. 7). Medela provides a Tender Care™ Hydrogel Pad that can provide instant cooling relief but they are too large to be comfortable, contain allergens that cause itching/irritation, and do not create an ideal moist wound healing environment. (https://www.medela.us/breastfeeding/products/breast-care/tender-care-hydrogel-pads-box-of-4). Lansinoh Soothies® are also meant to provide instant relief with cold feeling but these also have irritating components and do not mimic the environment needed for quick healing (https://lansinoh.com/products/soothies-gel-pads). LilyPadz® Nursing Pads are designed to “use pressure to control breast milk leakage” (https://lilypadz.com/product/lilypadz-single-pair/) (U.S. Pat. No. 6,200,195 https://patentimages.storage.googleapis.com/51/f5/ef/2d3b13b95f2eb7/US6200195.pdf). Medela Tender Care™ Hydrogel Pads, Lansinoh Soothies® and LilyPadz® are some examples of current dressings that are a simple covering for nursing nipples but which do not provide the appropriate advanced healing technologies to this clinical problem.

Therefore, an objective of the present invention is to provide wound healing strategies to the nursing mother. Another objective is to provide a wound healing dressing that is non-prescription, that is easy to apply, provides a non-toxic treatment and will not endanger the nursing newborn but also will not injure the mother's nipples with frequent removal and re-application.

### SUMMARY

The present invention is directed to embodiments of a dressing for a breast nipple. The ideal dressing creates a contained, moisture-rich environment needed to promote keratinocyte growth using non-allergic, non-toxic substances, adaptable to many breasts, spherical in shape, and may be frequently and non-injuriously removable as is consistent with the nursing frequency of newborns. The use of a hydrocolloid layer/non-adherent protective layer dressing will alleviate pain and allow for rapid healing. More specifically embodiments of the dressing are suitable for a nursing nipple, especially a traumatized nipple that suffers from redness and rawness of skin and more serious afflictions including but not limited to abrasions, fissures, and even full-thickness ulcers, which often then become a “petri dish” for infections like Staphylococcus aureus, Candida albicans, and even Pseudomonas aeruginosa or viral infections such as Herpes Simplex. The nursing breast suffering from such maladies presents a unique healing problem. The source of the malady, the nursing infant, cannot be turned away from nursing without adverse consequences. Nipple skin disruption can inhibit breastfeeding goals such as delivery of nutrients as well as anti-infective agents in mother's milk that are not found in bottle formulas. Thus, the American Academy of Pediatrics sets a 6-month goal for breastfeeding. Moreover, it is now recognized that human milk can protect against childhood obesity, autoimmune disorders like eczema and diabetes, and also improve a mother's recovery from childbirth. Furthermore, at least some infants have or develop allergies to bovine milk as well as synthetic nursing formulas. Nursing also cannot be temporarily suspended until the breast heals. Suspension of nursing will rapidly lead to milk production “drying” or mild production shut down of the mother's milk production tissues.

Embodiments of the dressing according to the invention are configured to fit over the nursing nipple with a minimum of discomfort that could be caused by overlaid clothing. These embodiments of the dressing may optionally be waterproof which can help reduce pain experienced by direct water application in a shower or bath. Embodiments of the dressing are thin and adhere to the breast skin with a minimum of adhesive.

Embodiments of the dressing incorporate a shaped sheet-like structure having an outer film that is preferably water impervious, and on one side of which is coated a hydrocolloid layer and a center portion coating of a non-adherent protective layer. The non-adherent protective layer either may overlay the hydrocolloid layer or may be coated directly onto a center portion of the film with the hydrocolloid layer surrounding this center portion so that for both versions the dressing may have a “donut” appearance with a filled-in center. The film may be a woven, nonwoven biologically acceptable, hypoallergenic fabric or film of a synthetic or natural polymer that may optionally be permeable to water and air or may optionally be impervious to water and air or may optionally be air permeable but impervious to water. The non-adherent protective layer may comprise a silicone material and/or a cellulose derivative material and/or a permeable or porous polyester material all of which may preferably be water absorbent and/or water swellable. The hydrocolloid layer may be a ring surrounding the non-adherent protective layer. Alternatively, the hydrocolloid layer may completely cover the film and the non-adherent protective layer may be overlaid on top of the hydrocolloid layer. These alternative constructions provide the appearance of a donut with the center being the non-adherent protective layer and the donut ring being a portion of the hydrocolloid layer. With either construction, the ring of the hydrocolloid layer forms an adherent seal with underlying skin but does not harm the skin with frequent removal/reapplication.

Embodiments of the dressing may have any acceptable circumferential shape such as square, rectangular, trapezoidal, elliptical or circular and the like and are typically circular in circumference and will cover the entire nipple and at least part of the areola. Preferably, embodiments are configured to cover the central part of the breast including the areola and nipple and most preferably may cover an area slightly larger than the nipple. The dressing may have a substantially flat sheet shape or may have a slight to moderate to large conical, cup or semi-spherical three-dimensional shape so that the dressing will fit and cover snuggly the surface of the nipple and at least part of the areola. The non-adherent protective layer is configured to cover the nipple and surrounding skin so that there is delicate contact, or minimal contact or substantially no contact with the eroded, ulcerated tissue. In the alternative providing substantially no contact, the non-adherent protective layer may be raised or elevated slightly above the surface of the ulcerated tissue. The slight elevation enables moisture transfer to the ulcerated tissue to promote healing The non-adherent protective layer enables ready removal of the dressing with substantially to essentially no damage to the injured nipple and areola. The centered middle portion of the dressing, i.e., the non-adherent protective layer, may optionally provide an adjustable space, colloquially termed herein as an optional igloo characteristic, to accommodate different nipple sizes so that long nipple sizes are not impinged by a short space of this centered middle portion. The donut and optional igloo characteristics mean that the non-adherent protective layer will not adhere or substantially adhere to the injured tissue so that removal of the dressing will not damage the injured tissue. The hydrocolloid ring adheres soundly to the skin around the periphery, thereby keeping out microbes like Staphylococcus aureus and Candida albicans that might take hold in the injured nipple skin.

### BRIEF DESCRIPTION OF DRAWINGS

FIG. 1 depicts an embodiment example of the dressing.

FIG. 2 depicts the front view of the dressing with outlines for cutting.

FIG. 3A depicts the side view of the dressing and shows the conical shape of the dressing.

FIG. 3B depicts the side view of the dressing with conical shape and extended center to accommodate nipple size.

FIG. 4 depicts the injured nursing breast at 6 weeks post-partum.

FIG. 5A depicts dressing examples in anterior, a larger dressing size with a silicone based center.

FIG. 5B depicts a dressing example in anterior, a medium size dressing with a cellulose acetate center.

FIG. 5C depicts a dressing example in anterior, a small dressing size with a silicone-based center.

FIG. 6A depicts an example in posterior, a larger dressing size with a silicone based center.

FIG. 6B depicts an example in posterior, a medium size dressing with a cellulose acetate center.

FIG. 6C depicts an example in posterior a small dressing size with a silicone-based center.

FIG. 7 depicts an over-the-counter hydrocolloid dressing (Band-Aid® Hydro Seal®), https://www.band-aid.com/products/hydro-seal-wound-care

### DEFINITIONS

Unless defined otherwise, all technical and scientific terms used herein have the same meaning as commonly understood by a person of ordinary skill in the art.

As used in the specification and the appended claims, the singular forms “a,” “an” and “the” include plural referents unless the context clearly dictates otherwise.

the term “may” in the context of this application means “is permitted to” or “is able to” and is a synonym for the term “can.” The term “may” as used herein does not mean possibility or chance.

The term and/or in the context of this application means one or the other or both. For example, an aqueous solution of A and/or B means an aqueous solution of A alone, an aqueous solution of B alone and an aqueous solution of a combination of A and B.

The term “about” is understood to mean ±10 percent of the recited number, numbers or range of numbers.

“Substantially” and “substantial” as the terms are used herein mean completely or almost completely; for example, removal substantially without damage means that the removal does not tear, injure or otherwise pull blood clots, scabs or tissue that is healing away from the surrounding skin.

All numerical amounts are understood to be modified by the word “about” unless otherwise specifically indicated. Unless otherwise indicated, all measurements are understood to be made at 25° C. and at ambient conditions, where “ambient conditions” means conditions under about one atmosphere of pressure and at about 50 percent relative humidity. All such weights as they pertain to listed features are based on the application level unless otherwise specified.

“Kit,” as used herein, means a packaging unit comprising a plurality of components. An example of a kit is, for example, a dressing with a release sheet. The package unit may have printed on its outer surface various outlines for fitting the dressing to differing sizes of breast/areolas/nipples. The dressing may be cut with a scissors by following an outline appropriate for the mother's breast. Alternatively, differing sizes of dressings may be packaged separately and coordinated with the nursing mother's breast cup size.

The molecular weight of a component may be expressed as a weight average molecular weight and given as KDa, kilodaltons or MDa, megadaltons.

### DETAILED DESCRIPTION

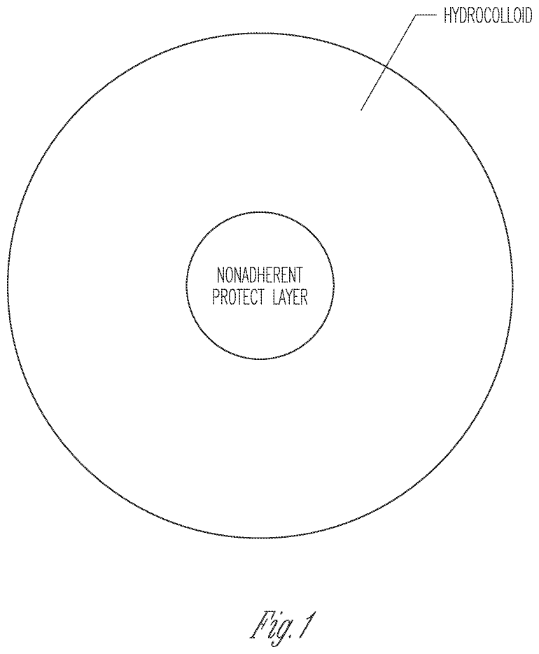

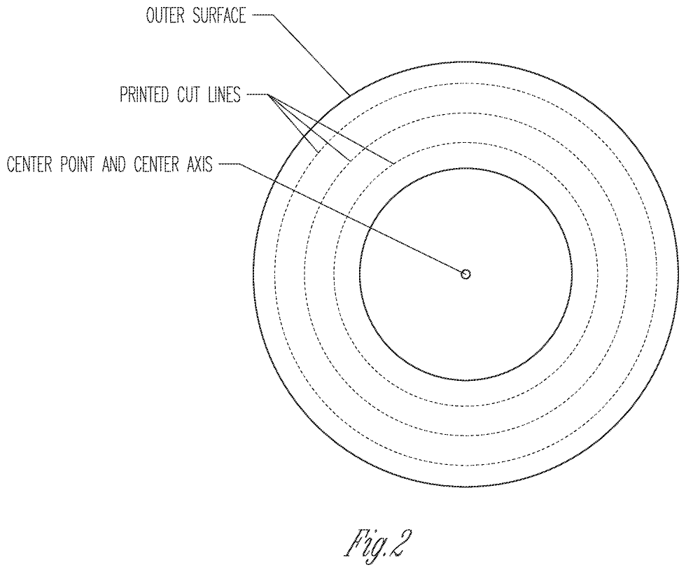

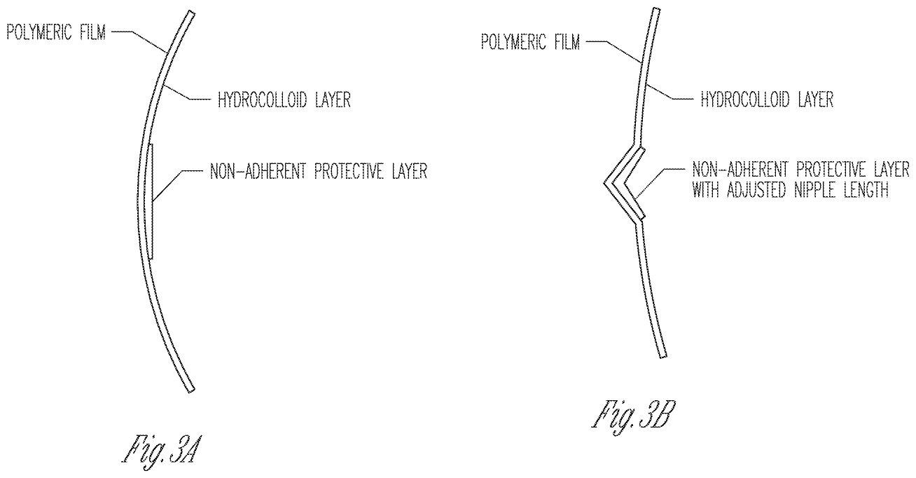

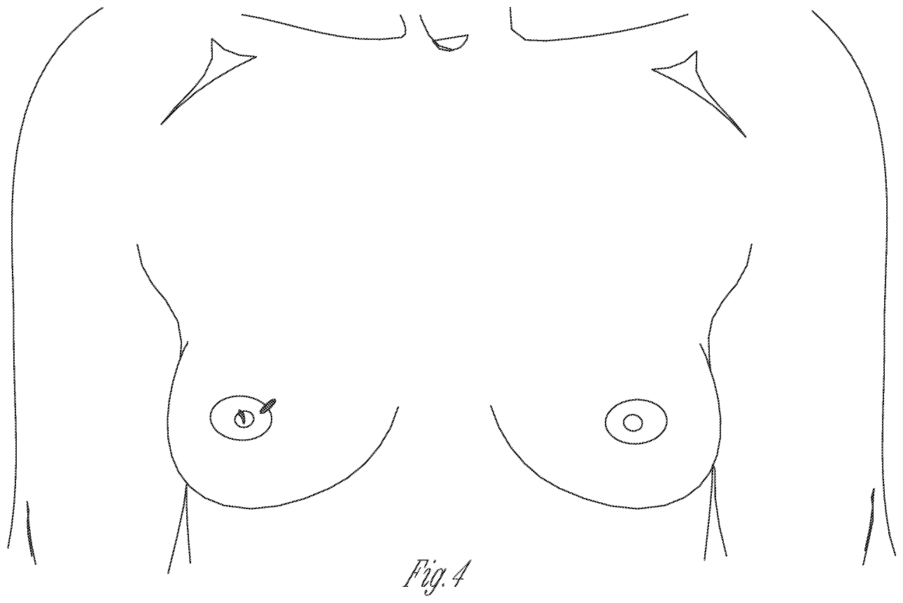

The developments of the invention are directed embodiments of a hypoallergenic igloo and/or donut dressing for the nursing breast. These embodiments fit snuggly on the nursing breast and through their hypoallergenic and hydrocolloid characteristics promote rapid healing of roughened nipple, a full-thickness wound, eroded, ulcerated and/or lacerated areola and similar difficulties affecting the nipple of a nursing breast (FIG. 1).

The maintenance of a protected moist environment of this tissue promotes its rapid healing through optimum ambience and autolytic debridement, which in turn decreases the pain experienced by the nursing mother. The protected moist environment for a wound may be typically achieved with bandages but a unique problem is associated with ulcerated and/or lacerated nipples. The bandage needs to be removed frequently, and removal typically reinjures the tissue when bandages known in the art are used. Like many of the other OTC breast bandages available, instructions to users would entail use of the bandage for up to 24 hours after opening the package.

These and other problems are solved by the present invention. The embodiments of the present invention include the hypoallergenic igloo and/or donut dressing. The dressing has inner and outer surfaces and an outer edge portion along its outer perimeter. The outer edge portion covers and may be adjacent to the border of the areola and the rest of the breast. Preferably, the outer edge portion covers much of the areola—large enough to encase the involved skin entirely but not so large that the dressing is cumbersome. The dressing must be manipulated several times daily for rapid and easy-access nursing, sometimes even in the dark in the middle of the night and thus the dressing may at some point be combined with a specific undergarment to fit the dressing inside. More preferably, the outer edge portion covers much of the areola and provides a circumference approximately in that range. An exemplary outer edge portion is arranged to cover the nipple and at least a part of the areola.

The dressing comprises on its inner side a centered, non-adherent protective layer having a retentive moisture content. The centered non-adherent protective layer is configured in area and shape to cover the areola with the nipple centered in the areola. The outer perimeter shape of the non-adherent protective layer is typically circular and is three dimensionally shaped as a conical, cup or semi-spherical configuration according to the three dimensional shape of the dressing. The non-adherent protective layer is arranged on the hydrocolloid layer to cover the nipple and surrounding skin. The non-adherent protective layer is configured to be non-adherent so that when the dressing is removed from the breast, the injured nipple and areola are not re-injured. This ability is accomplished through use of a composition for the non-adherent protective layer that easily releases from tissue without pulling away healing tissue, scabs, blood clots and viscous body fluid. The composition of the non-adherent protective layer may comprise at least a silicone material, a cellulose derivative material and/or a permeable or porous polyester material. These materials comprise water absorptive and water retentive properties.

While the dressing may be configured with any outer perimeter shape including a circular, elliptical, square, rectangle, trapezoid or other polygon shape, the preferred perimeter shape is circular. The dressing may also have any three dimensional shape ranging from a flat sheet to a conical, cup or semispherical shape. The preferred three-dimensional shape is conical, cup or semispherical designed to fit snuggly and completely onto the shape of a nursing nipple. The conical, cup or semi-spherical shape should fit a variety of breast cup sizes from A to triple D and a variety of nipple sizes. The custom fit for the breast may be accomplished through an arrangement in which the film of the dressing may have on its outer surface a series of printed approximately circular outlines and optional printed numbers associated with the circular outlines indicating the approximate diameters of the circles. The circular outlines enable the dressing to be cut to an appropriate dimension to correspond to the desired area of the breast to cover. The sizes of nipples also vary so that a design of the dressing to fit may include customization of that element of size. The customization may be accomplished by configuring the center portion along the center axis of the dressing to have an accordion pull-out arrangement so that a segment of this center portion may be expanded to accommodate larger nipples. Alternatively, this segment of the center portion of the dressing may be adapted so as to provide a series of dressing having a series of segment lengths along the center axis relative to and perpendicular to the plane on which the outer perimeter of the dressing would rest. These lengths would provide a series of dressings with comfortable ranges of nipple sizes.

The hydrocolloid layer comprises a hypoallergenic carbohydrate which is optionally cross linked and/or a hydrophilic olefin polymer. Hydrocolloids are partly water-soluble, natural, or synthetic, polymers which form viscous solutions or gels. It is often advantageous for the hydrocolloid to be gelatin and/or collagen. Exemplary hydrocolloids include such carbohydrates as optionally cross linked polysaccharides including but not limited to cellulosic derivatives, guar gum derivatives, pectin derivatives, locust bean derivatives, xanthan gum derivatives, mannan derivatives, galactomannan derivatives, xyloglucan derivatives, arabinoxylan derivatives and similar polysaccharide derivatives as well as synthetic hydrogel polymers such as but not limited to (meth)acrylate hydroxyesters, vinyl alcohol, allyl alcohol and similar hydrogel polymers and any combination thereof. Non-allergenic additives such as polyolefins may be present to promote strength and strong amalgamating or binding of the carbohydrate and/or hydrogel polymer substances. The hydrocolloid sheet may have on or as its outer surface a microporous polymeric sheet that preferably substantially to essentially completely minimizes vaporization and evaporation of components from the hydrocolloid sheet, such as but not limited to water. Exemplary hydrocolloids are described in U.S. Pat. No. 9,782,512.

The hydrocolloid may be formed by extrusion, solvent spin casting or melt die formation into particulate material such as fibers, filaments, particles and/or microparticles. The particulate material may be combined as woven, non-woven, agglomerated and/or tacked particulate and formed as a layer of such material on the polymeric film serving as the outside surface of the dressing. The polymeric film may be PVC, polyethylene or polyurethane or latex and is formed into a continuous film by casting or extrusion formation. The hydrocolloid layer may be deposited on the polymeric sheet in a semi to substantially solid state so that the hydrocolloid binds to the surface of the polymeric film. The polymeric film with hydrocolloid layer may be cut or otherwise shaped into the appropriate dimensions for the dressing. The outer surface of the polymeric film may be optionally painted with glow-in-the-dark paint such as using zinc sulfide or other compounds described below.

The non-adherent protective layer includes a material that is water absorptive and water retentive so that healing of the protected, injured tissue is promoted. This material may at least be a silicone material, a carbohydrate derivative material or a polyester material.

The silicone material may at least be a hypoallergenic polydimethylsilicone of the MDTQ structure with minor T and Q links and a weight average molecular weight of between about 1 KDa and about 1 MDa, preferably about 5 KDa to about about 500 KDa, more preferably about 5 KDa to about 200 KDa. Alternatively, or additively, the silicone material may at least be an organosilicone material composed of a polymer of alkylsiloxane and ester and/or polyol moieties. The weight average molecular weight of the organosilicone material may have the same range and preferred ranges as the MDTQ polydimethyl

This silicone material may be configured as a non-structured agglomeration or gel, as a contiguous three-dimensional solid, as spun or extruded fibers and/or microfilaments in a configuration of a three-dimensional woven or non-woven fabric. The silicone material is constructed to have flexible and soft properties, may be silicone gel, and may optionally be mixed with a high average molecular weight hydrogel. The hydrogel may be a moderate to high weight average molecular weight polyol such as a polyethylene glycol. Alternatively, and/or additionally, the silicone may be a silicone hydrogel copolymer of silicone polymer segments and hydrophilic (meth)acrylate ester polymer and/or vinyl alcohol and/or allyl alcohol segments or a silicone polymer with hydrophilic (meth)acrylate ester side chains and/or vinyl alcohol and/or allyl alcohol side chains. The hydrophilic (meth)acrylate ester is formed of polyol alcohols as the esterifying alcohol. The polydimethylsilicone and polyol mixture and/or silicone hydrogel may retain and/or contain water at a percentage of at least 20 percent of the weight of the mixture, preferably up to 70 percent of its weight as water. A silicone facing layer has significant advantages over wound dressings that rely on glue-type adhesive to secure a dressing to a wound. Silicone gels are particularly non-adherent to wounds but significantly adherent to surrounding skin. Because they are unaffected by heat, the dressings retain their non-adherent properties. The silicone layer more fully distributes its adhesion force so the peeling strength does not strip epidermal cells when the dressing is removed.

A cellulose derivative material may also function as the non-adherent protective layer. The cellulose derivative may be a cellulose ester such as but not limited to cellulose acetate, cellulose acetate butyrate, cellulose propionate, cellulose triacetate, cellulose acetate propionate and the like. The cellulose derivative may also be a cellulose ether such as but not limited to ethyl cellulose, ethyl methyl cellulose, hydroxyethyl cellulose, hydroxylpropyl cellulose hydroxypropyl methyl cellulose and the like. The cellulose ethers that display water solubility may be mixed with water insoluble cellulose esters and/or cellulose ethers. The cellulose derivative as the non-adherent protective layer may be configured as a non-structured agglomeration or gel, as a contiguous three-dimensional solid, as spun or extruded fibers and/or microfilaments in a configuration of a three-dimensional woven or non-woven fabric. The cellulose derivative has non-adherent, water absorptive and water retentive characteristics. A preferred cellulose derivative material is cellulose acetate.

Preferably, the non-adherent protective layer of cellulose derivative may be formed of woven or non-woven fibers and then optionally finished with a siloxane material (0.01 to 0.0001%). Either dry spinning or wet spinning can be used to form the non-adherent protective layer of cellulose derivative fabric using spinnerets with 50 to about 250 openings to form woven and/or non-woven threads. Subsequent to fabric forming and needling, the fabric may undergo optional additional treatments such as washings. For example, the fabric may be washed and treated with an additive providing further non-adherent properties such as a siloxane described above. Following siloxane treatment, the fabric is optionally washed to remove excess silicone and dried, resulting in a final siloxane percentage in the range of 0.01% to 0.0001%. The fabric may then be folded, cut, and in this case, placed in the center of the bandage. See EP0940147A2 and references cited therein for further examples of non-adherent cellulose derivatives.

The non-adherent protective layer of polyester derivative may be formed into fibers that may be combined as a woven or non-woven fabric having water porous properties. Alternatively, the polyester derivative may be cast as a contiguous polymeric sheet that may be rendered water porous or water permeable by microscoring with electric spark or microneedles or by casting with solvent that will produce pores by evaporation as the contiguous sheet is cast. The fabric or sheet may be combined with water absorbent material such as a hydrogel or cellulose derivative fibers to form the non-adherent protective layer. The polyester fabric or sheet may be arranged as a sleeve, bag or sheet into or onto which the entwined hydrogel and/or cellulose derivative fibers are inserted or layered. The fabric or sheet enables transfer of water into the entwined fibers within the sleeve or bag or on the sheet. The fabric or sheet is non-adherent so that it does not allow binding or adherence of healing tissue and the non-adherent protective layer especially upon separation of the layer from the healing tissue.

The polyester derivative may be a pharmaceutically acceptable water-insoluble polymer of diacids such as phthalic acid, terephthalic acid, adipic acid, succinic acid, glutaric acid, and similar alkyl dicarboxylic acids, and diols such as ethylene glycol, propylene glycol, butane diol, hexane diol and similar alkyl diols. The weight average molecular weight of the polyester derivative may range from about 5 KDa to 50 KDa.

The non-adherent protective layer may be fitted or placed onto the center portion of the hydrocolloid layer with outer polymeric film to form the parts of the dressing. Alternatively, the non-adherent protective layer may be formed directly onto the polymeric film and the hydrocolloid layer formed as a ring surrounding this center portion of non-adherent protective layer. In this alternative arrangement, the non-adherent protective layer may include additional water absorptive and water retentive material as an inner layer between the polymeric film and the non-adherent layer. This arrangement may be adapted to the silicone, cellulose and polyester compositions of the non-adherent protective layer and is preferred for the polyester composition.

As is appropriate for assembly techniques, the polymeric film may be pressed with optional heat at its center portion before or after application of the hydrocolloid layer to form the telescoping or extended center segment for nipple fitting. Simultaneous or sequentially the polymeric film may also be pressed with optional heat to form the desired overall three dimensional shape of the dressing fitting over the breast segment as described above. A preferred method of assembly may be addition of the hydrocolloid and non-adherent protective layers following the double pressing of the polymeric sheet. However, these layers may also be combined with the polymeric sheet before it is pressed with optional heating to form the nipple fitting segment and the desired overall three dimensional shape.

Antimicrobial additives may be optionally combined with the non-adherent layer. The antimicrobial additives may be bactericidal or bacteriostatic agents as well as antiviral agents. Examples include but are not limited to mupirocin, tetracyclines, beta lactams, macrolides, aminoglycosides, antifungals and nucleoside analogues. The control of pH may also be managed by incorporation of mild buffering agents within the silicone layer. The buffering agents are preferably configured to provide a pH environment substantially the same as the skin of the areola and breast. Additionally, and/or alternatively, topical soothing agents, antimicrobial agents, antiviral agents, emollients, balms, cremes, probiotic dressings, and related agents that are pharmaceutically acceptably may be applied to the injured area of the breast before application of the dressing.

The moisture retaining properties of the hydrocolloid layer and non-adherent protective layer bathe the irritated skin and nipple with a constant supply of moisture in the form of water. The hydrocolloid is capable of absorbing water and retaining significant quantities of water relative to the weight of the hydrocolloid. The quantities of water absorbed and/or retained by the hydrocolloid may range up to about 70 percent of the average weight of the hydrocolloid substances, preferably up to about 60 percent, more preferably up to about 50 percent.

As described above, the dimensions of the dressing are designed to cover snuggly the skin of the breast including but not limited to the areola and its nipple, and preferably only the areola and nipple. Typical and usual dimensions of the non-adherent protective layer of the dressing range up to approximately 5 to 7 cm in each of two flat configuration directions or as the diameter of a circular shape to cover the average size of women's nipples (21-36 mm) with the total diameter for the dressings being a diameter of up to about 5 cm to cover the entire nipple ranging 35-65 mm. Preferably, the flat or circular dimension or diameter ranges up to 8 cm, especially preferably up to 4 to 5 cm. The more preferable dimension is customized to size by cutting the outer perimeter of the dressing as described above. Preferably, the custom dimension is approximately the size of a U.S. quarter or fifty cent coin such as up to about 8 cm, preferably up to about 5 cm, more preferably up to about 2 or 3 cm. Most preferably, the circular dimension of the dressing before customization is slightly to somewhat larger than the average outer circumferential diameter of the areola and nipple such as but not limited to up to about 10 cm. The dressing of this most preferable circular configuration has on its outer surface the printed circular outlines described above. This preferred embodiment of the dressing may be cut to custom size to fit on the injured area of the breast such as the areola and nipple. Additionally, a most preferable dressing may be manufactured to approximate the coin size described above. The thickness of the sheet may range up to about 5 mm, preferably up to about 3 mm, more preferably up to about 2 mm so that the dressings are unbulky and comfortable.

The non-adherent protective layer is configured with a shape to fully cover the areola and nipple of the irritated and/or injured breast. The non-adherent protective layer typical has a circular configuration and may have a diameter of up to 1 to 5 cm.

Embodiments of the dressing may have an optional non-tacky hypoallergenic adherent substance coating a portion of the outer edge of the inner surface of the dressing so that the dressing may adhere snuggly to the breast and may be retained on the breast under ordinary pressure resulting from brushing by clothing or by handling but will easily release with moderate removal pressure. The hypoallergenic adherent coating would be adapted to remain on the outer portion of the inner surface when the dressing is configured to be custom cut to size. The adherent coating would not extend into and/or close to the silicone layer configured to cover the areola and nipple.

Embodiments of the dressing may further include a release sheet covering the inner surface of the dressing. The release sheet may be made of an organic polymeric or cellulosic derivative material. The release sheet may be removed without damage to the hydrocolloid and, non-adherent protective layers. The release sheet is similar to the release sheets commonly present with band-aids.

Embodiments of the dressing may be applied proactively to avoid or minimize irritation of the nursing breast. Embodiments of the dressing may also be applied post injury or post irritation to promote healing of the irritated and/or injured breast tissue.

Embodiments of the dressing may be stored in sterile foil or plastic wrap packages. The package may be opened by the nursing mother, the release sheet removed if present and the dressing applied to cover the at least the injured portion of the breast. Alternatively, and/or additionally, the dressing may be cut to custom size before application as described above. With a conical, cup or semi-spherical shape, the dressing may be self-orienting so that the silicone layer is centered over the areola. With a flat shape the silicone layer may be centered by holding a printed dot on the outer side of the dressing directly over the breast nipple. The printed dot will be centered in the middle of the silicone layer. Pressing the optional non-tacky adherent edge of the dressing onto the skin will enable a snug fit of the dressing on the breast. Once opened, the bandage will be safe to use for at least 24 hours.

Glow-in-the-dark technologies in the form of anterior stickers or paints could be applied to the dressing if deemed safe and pharmaceutically acceptable in the form of zinc sulfide, calcium sulfide, strontium aluminate or alkaline earth metal silicate, e.g., potassium, calcium, zinc or strontium silicate to allow nursing mothers at night to visualize the dressings.

### EXAMPLES

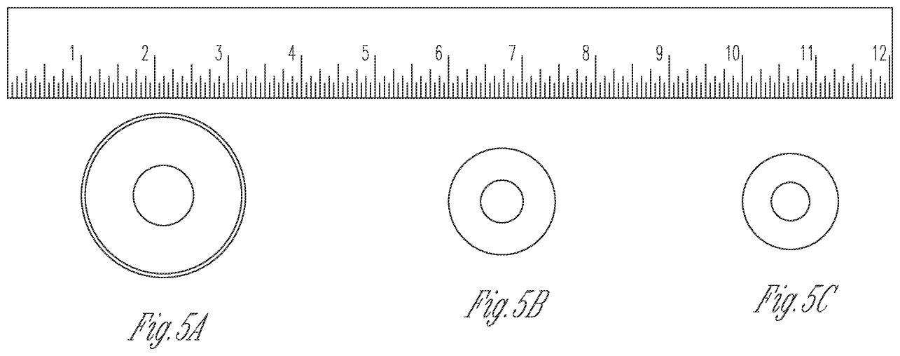

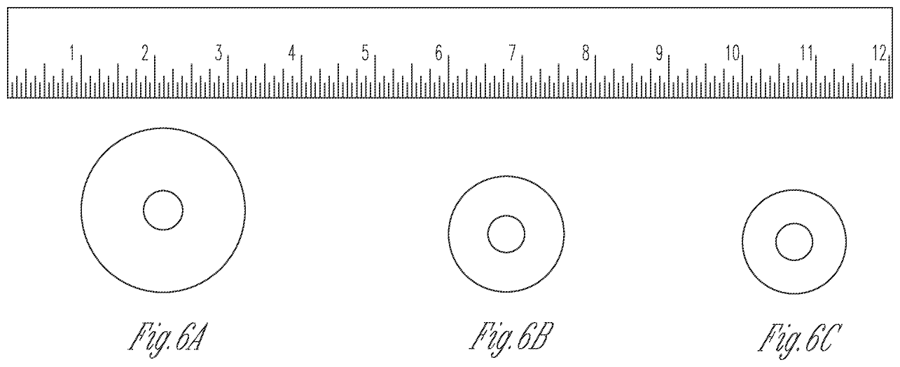

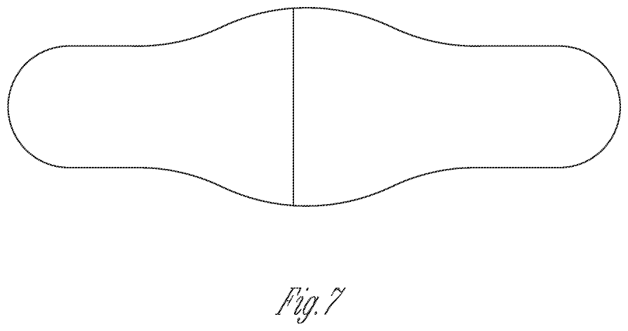

The dressings shown by FIGS. 5 (anterior view) and 6 (posterior view) are composites with a “donut ring” of adherent hydrocolloid ring which forms the contact with the breast/areola tissue. The center “active pouch” which cups the irritated or wounded nipple skin is formed from a non-adherent protective material such as a silicone or cellulose acetate material. These dressings may be formed of various cup sizes (A, B, and C).

### REFERENCES

1. What are the recommendations for breastfeeding? Eunice Kennedy Shriver National Institute of Child Health and Human Development. https://www.nichd.nih.gov/health/topics/breastfeeding/conditioninfo/recommendations. Accessed Dec. 1, 2018.
2. Odom E C, Li R, Scanlon K S, Perrine C G, Grummer-Strawn L. Reasons for earlier than desired cessation of breastfeeding. Pediatrics 2013; 131:e726-32.
3. Eglash A, Montgomery A, Wood J. Breastfeeding. 2008; 54:343-411.
4. McCann M. F., Baydar N., Williams R. L. Breastfeeding attitudes and reported problems in a national sample of WIC participants. J. Hum. Lact. 2007; 23:314-324. doi: 10.1177/0890334407307882.
5. Wagner E. A., Chantry C. J., Dewey K. G., Nommsen-Rivers L. A. Breastfeeding concerns at 3 and 7 days postpartum and feeding status at 2 months. Pediatrics. 2013; 132:865-875. doi: 10.1542/peds.2013-0724.
6. McClellan H. L., Hepworth A. R., Garbin C. P., Rowan M. K., Deacon J., Hartmann P. E., Geddes D. T. Nipple pain during breastfeeding with or without visible trauma. J. Hum. Lact. 2012; 28:511-521. doi: 10.1177/0890334412444464
7. Wüthrich B. Minimal Variants of Atopic Eczema. Handbook of Atopic Eczema.:74-83. doi:10.1007/3-540-29856-8_8.
8. Ziemer M M, Pigeon J G. Skin Changes and Pain in the Nipple During the 1st Week of Lactation. Journal of Obstetric, Gynecologic & Neonatal Nursing. 1993; 22(3):247-256. doi:10.1111/j.1552-6909.1993.tb01806.x.
9. Breastfeeding Support. Milk Matters Infant Feeding Solutions. http://milkmatters.org.uk/services-offered/postnatal-feeding-support/. Accessed Nov. 24, 2018.
10. Barrett M E, Heller M M, Stone H F, Murase J E. Dermatoses of the Breast in Lactation. Dermatologic Therapy, 2013; 26:331-336.
11. Buck M L, Amir L H, Cullinane M, Donath S M. Nipple Pain, Damage, and Vasospasm in the First 8 Weeks Postpartum. Breastfeeding Medicine, 2014; 9(2):56-62.
12. Righard, L.; Made, M. O. Sucking technique and its effect on success of breastfeeding. Birth 1992, 19, 185-189.
13. Kent J. Ashton E, Hardwick C, et al. Nipple Pain in Breastfeeding Mothers: Incidence, Causes and Treatments. International Journal of Environmental Research and Public Health. 2015; 12(10):12247-12263. doi:10.3390/ijerph121012247.
14. McKechnie A. C., Eglash A. Nipple shields: A review of the literature. Breastfeed. Med. 2010; 5:309-314. doi: 10.1089/bfm.2010.0003.
15. Lv X, Feng R, Zhai J. A combination of mupirocin and acidic fibroblast growth factor for nipple fissure and nipple pain in breastfeeding women: protocol for a randomised, double-blind, controlled trial. BMJ Open. 2019; 9(3). doi:10.1136/bmjopen-2018-025526
16. Dennis C-L, Jackson K, Watson J. Interventions for treating painful nipples among breastfeeding women. Cochrane Database of Systematic Reviews. 2014. doi: 10.1002/14651858.cd007366.pub2.
17. Melli M S, Rashidi M R, Delazar A, et al. Effect of peppermint water on prevention of nipple cracks in lactating primiparous women: a randomized controlled trial. International Breastfeeding Journal. 2007; 2(1):7. doi:10.1186/1746-4358-2-7.
18. Dennis C L, Schottle N, Hodnett E, McQueen K. An all-purpose nipple ointment versus lanolin in treating painful damaged nipples in breastfeeding women: A randomized controlled trial. Breastfeed Med. 2012; 7:473-9
19. Jackson K T, Dennis C L. Lanolin for the treatment of nipple pain in breastfeeding women: A randomized controlled trial. Matern Child Nutr. 2017; 13:e12357
20. Morland-Schultz K, Hill P D. Prevention of and Therapies for Nipple Pain: A Systematic Review. Journal of Obstetric, Gynecologic & Neonatal Nursing. 2005; 34(4):428-437. doi:10.1177/0884217505276056.
21. Gungor A N C, Oguz 5, Vurur G, et al. Comparison of Olive Oil and Lanolin in the Prevention of Sore Nipples in Nursing Mothers. Breastfeeding Medicine. 2013; 8(3):334-335. doi:10.1089/bfm.2012.0131.

### SUMMARY STATEMENTS

The inventions, examples and results described and claimed herein may have attributes and embodiments include, but not limited to, those set forth or described or referenced in this application.

All patents, publications, scientific articles, web sites and other documents and ministerial references or mentioned herein are indicative of the levels of skill of those skilled in the art to which the invention pertains, and each such referenced document and material is hereby incorporated by reference to the same extent as if it had been incorporated verbatim and set forth in its entirety herein. The right is reserved to physically incorporate into this specification any and all materials and information from any such patent, publication, scientific article, web site, electronically available information, textbook or other referenced material or document. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

The written description of this patent application includes all claims. All claims including all original claims are hereby incorporated by reference in their entirety into the written description portion of the specification and the right is reserved to physically incorporated into the written description or any other portion of the application any and all such claims. Thus, for example, under no circumstances may the patent be interpreted as allegedly not providing a written description for a claim on the assertion that the precise wording of the claim is not set forth in haec verba in written description portion of the patent.

While the invention has been described in conjunction with the detailed description thereof, the foregoing description is intended to illustrate and not limit the scope of the invention, which is defined by the scope of the appended claims. Thus, from the foregoing, it will be appreciated that, although specific nonlimiting embodiments of the invention have been described herein for the purpose of illustration, various modifications may be made without deviating from the scope of the invention. Other aspects, advantages, and modifications are within the scope of the following claims and the present invention is not limited except as by the appended claims.

What is claimed is:

1. A dressing comprising a hydrocolloid layer on a polymeric sheet and a non-adherent protective layer on the center segment of the hydrocolloid layer contiguous with the polymeric sheet or the non-adherent protective layer on the center segment of the polymeric sheet with the hydrocolloid layer ringing the non-adherent protective layer on the polymeric sheet, the dressing having an outer edge, an inner surface formed by the hydrocolloid and non-adherent protective layers and an outer surface formed by the polymeric sheet.

2. A dressing according to claim 1, wherein at least the non-adherent protective layer has a three dimensional shape adapted to provide delicate contact, or minimal contact or substantially no contact with breast tissue when the dressing is applied to a breast.

3. A dressing according to claim 1, wherein the hydrocolloid comprises at least a hypoallergenic carbohydrate and/or a hydrophilic synthetic hydrogel or a combination thereof.

4. A dressing according to claim 1 wherein the non-adherent protective layer is hypoallergenic, has a thickness of at least about one millimeter and comprises a silicone material, a cellulose derivative material, a polyester material or a combination thereof.

5. A dressing according to claim 4 wherein the non-adherent protective layer is capable of containing up to at least about 40 percent by weight water relative to the total weight of the non-adherent protective layer.

6. A dressing according to claim 4 wherein the non-adherent protective layer has an average length and width or diameter each of up to about 35 mm to about 65 mm.

7. A dressing according to claim 3 wherein the hydrocolloid layer has average length and width dimensions or a diameter of up to about 6-7 centimeters and a thickness of up to about 40 mm.

8. A dressing according to claim 7 wherein the non-adherent protective layer has an average perimeter dimension of up to about 1 to 2 centimeters.

9. A dressing according to claim 7 wherein the hydrocolloid layer has a circular outer edge describing a diameter of up to about 6 to 7 centimeters and the non-adherent protective layer has a length and width describing a circular circumference of a radius of up to about 5 centimeters.

10. A dressing according to claim 1 wherein the hydrocolloid layer is capable of absorbing water up to about 70 percent of the weight of the hydrocolloid.

11. A dressing according to claim 4 wherein the non-adherent protective layer is a silicone material, is hypoallergenic and the silicone material has minor T and Q links and flexible, soft properties.

12. A dressing according to claim 11 wherein the silicone material is a silicone MDTQ hydrogel or is a silicone copolymer with dimethylsilicone segments and hydrophilic (meth)acrylate ester or vinyl alcohol or allyl alcohol polymer segments or a dimethylsilicone polymer with side chains of and hydrophilic (meth)acrylate ester or vinyl alcohol or allyl alcohol polymer.

13. A dressing according to claim 4 wherein the silicone material, cellulose derivative material and/or polyester material is mixed with a high average molecular weight hydrogel to provide a non-adherent protective layer-hydrogel combination.

14. A dressing according to claim 13 wherein the non-adherent protective layer combination retains and/or contains water at a percentage of up to 60 percent of the weight of the mixture.

15. A dressing according to claim 14 wherein the combination maintains its water percentage at least for 10 hours while in contact with ambient condition.

16. A dressing according to claim 1 wherein the outer surface of the hydrocolloid layer is covered by a microporous polymeric sheet.

17. A dressing according to claim 1 wherein the outer edge of the inside surface of the hydrocolloid layer is coated with a non-tacky hypoallergenic adherent coating for binding the dressing to skin.

18. A dressing according to claim 1 further comprising a release sheet polymer covering the inner surface of the dressing.

19. A dressing according to claim 1 wherein the dressing is suitable as a protective covering for a nursing nipple.

20. A dressing according to claim 1 wherein the dressing at least partially alleviates a painful nursing nipple.

21. A dressing according to claim 1 wherein the outer surface of the dressing has a series of printed circular outlines designed for cutting the dressing to a custom size for a nursing breast.

22. A package containing a dressing of claim 1 contained in a container, sack or bag.

23. A method for use of a dressing according to claim 1 comprising removing the dressing from the package and if a release sheet polymer covers the inner side of the dressing, removing the release sheet polymer, optionally cutting the dressing to custom size and applying the inner side of the dressing to a nursing breast.

24. A method according to claim 23 wherein the non-adherent protective layer is centered on the breast nipple.

25. A method according to claim 24 wherein at least the outer periphery of the hydrocolloid layer of the dressing has a mildly adhesive edge.

26. A dressing according to claim I wherein the dressing has a center axis perpendicular to the plane established by the perimeter of the dressing and the center axis is length adjustable, the length adjustment providing an adaptation of the non-adherent protective layer to accommodate variations of nipple sizes.

27. A dressing according to claim 26 wherein adjustment of the length of the center axis provides a set of non-adherent protective layer lengths along the center axis.

28. A dressing according to claim 2 wherein the three dimensional shape of the non-adherent protective layer is adapted to be raised or elevated slightly above the surface of the tissue.
